(19) United States
(12) Patent Application Publication
(10) Pub. No.: US 2026/0198957 A1
(43) Pub. Date: Jul. 16, 2026

(54) HANDHELD RESECTION MEDICAL DEVICE WITH INTEGRATED PARTICULATION SYSTEM

(71) Applicant: Arthrex, Inc., Naples, FL (US)

(72) Inventors: Bryan Cannon, Miami, FL (US); Robert Benedict, Naples, FL (US); Jacek Kluzik, Munich (DE)

(73) Assignee: Arthrex, Inc., Naples, FL (US)

(21) Appl. No.: 19/566,262

(22) Filed: Mar. 13, 2026

Related U.S. Application Data
(63) Continuation of application No. PCT/US2024/046916, filed on Sep. 16, 2024.
(60) Provisional application No. 63/582,760, filed on Sep. 14, 2023.

(51) Int. Cl. A61B 17/32

(52) U.S. Cl. CPC A61B 17/32002; A61B 2217/005

(57) ABSTRACT

A handheld rotary medical device configured to harvest material from a patient and create particulates of the material within an inner drive shaft is disclosed. As such, material collected at a distal end of an outer shaft is processed via an inner drive shaft working member before the material is evacuated out of the outer shaft.

### CROSS-REFERENCE TO RELATED APPLICATION

This application is a continuation of and claims priority to International Application No. PCT/US2024/046916 filed Sep. 16, 2024, which claims priority to U.S. Provisional Application No. 63/582,760, filed Sep. 14, 2023, each of which are incorporated herein by reference in their entirety.

### BACKGROUND

The disclosure relates to handheld medical devices, and more particularly, to handheld, arthroscopic medical devices having rotatable inner shafts configured to be partially inserted into patients for tissue removal.

Arthroscopic medical devices include rotary driven shavers and burrs configured to remove material, such as, but not limited to, bone, cartilage and tissue, from a patient. Conventional arthroscopic medical devices often include shavers or burrs configured to excise material at distal ends of the devices and transmit the harvested material to a collection system positioned outside of the device. Harvested material is processed, if at all, downstream of the collection system.

### SUMMARY

A handheld rotary medical device configured to excise material from a patient and create particulates of the material within an inner drive shaft is disclosed. In at least one embodiment, the device may include a rotatable inner drive shaft positioned within an elongated, tubular, outer shaft, whereby the inner drive shaft includes an inner drive shaft working member configured to reduce excised material within a channel created by the outer shaft from material, such as, but not limited to, bone, cartilage and tissue, drawn through a distal opening in the outer shaft before the reduce excised material is evacuated proximally therefrom. As such, the material collected at a distal end of an outer shaft is processed via an inner drive shaft working member before the material is evacuated and sent into a material collection system.

In at least one embodiment, the handheld rotary medical device may include an inner drive shaft and an elongated, tubular, outer shaft encapsulating the inner drive shaft such that the inner drive shaft is positioned within the outer shaft. The device may include an inner drive shaft working member at a distal end of the inner drive shaft, whereby a distalmost point of the inner drive shaft working member resides within a channel created by the outer shaft and is offset proximally from a distal end of the outer shaft. In such configuration, the inner drive shaft working member resides within the outer shaft. The inner drive shaft working member may be configured to reduce excised material within the channel created by the outer shaft from tissue drawn through a distal opening in the outer shaft before the material is evacuated proximally therefrom. In at least one embodiment, the distalmost point of the inner drive shaft working member may reside within a channel created by the outer shaft generally in a midsection between distal and proximal ends of the outer shaft.

The inner drive shaft working member may be configured to process material received within the distal opening in the outer shaft. The inner drive shaft working member may have any appropriate configuration. In at least one embodiment, the inner drive shaft working member may be configured as a bulbous cone with one or more material engaging members on an outer surface thereof for engaging material, such as, but not limited to, bone, cartilage and tissue. The material engaging member on the outer surface of the inner drive shaft working member may be a plurality of burrs configured for engaging material. In another embodiment, the material engaging member may include a plurality of teeth extending radially outward from the outer surface of the inner drive shaft working member. In at least one embodiment, the plurality of teeth may be aligned into rows.

The device may include one or more openings in the inner drive shaft for capturing reduce excised material after passing the inner drive shaft working member in the channel created by the outer shaft. In at least one embodiment, the inner drive shaft opening may be near the distal end of the inner drive shaft and positioned proximal of the inner drive shaft working member.

The outer shaft working member may be positioned at a distal end of the outer shaft and configured to capture material so that the material can be evacuated proximally therefrom. The outer shaft working member may be a sharp freer configured to excise tissue. In another embodiment, the outer shaft working member may be a curette configured to excise tissue.

The device may include one or more material collection systems in fluid communication with the inner drive shaft to evacuate material through the inner drive shaft. In at least one embodiment, the material collection system may be an autologous tissue collector, such as, but not limited to a GRAFTNET system sold by Arthrex Inc., Naples, Florida.

The device may include one or more outer shaft bases which in turn may include one or more releasable connections configured to be releasably attachable to a handle of the medical device. The releasable connection may be any configuration enabling a releasable connection to be formed between the outer shaft and the handle of the medical device. The device may include one or more inner shaft bases which in turn may include one or more releasable connections configured to be releasably attachable to a handle of the medical device. The releasable connection may be any configuration enabling a releasable connection to be formed between the inner shaft and the handle of the medical device. Thus, the bases are configured to be releasably coupled directly or indirectly to a handle.

An advantage of this system is that the inner drive shaft working member may be configured to process material received within the distal opening in the outer shaft to a desired degree such that the material evacuated proximally is processed to a greater degree than if the material were only harvested at a distal end of the outer shaft and evacuated therefrom.

Another advantage of this system is that the system may include multiple inner drive shaft working members whereby two or more of the inner drive shaft working members may be configured to process material entering into the channel formed by the outer shaft to different degrees of processing before the material is evacuated from the shafts.

Yet another advantage of this system is that the system may include multiple inner drive shaft working members whereby two or more of the inner drive shaft working members may have different configurations, such as one inner drive shaft working member designed to process bone efficiently, one inner drive shaft working member designed to process cartilage efficiently and one inner drive shaft working member designed to process tissue efficiently.

Another advantage of this system is that the system may include multiple inner drive shaft working members may include inner drive shaft working member configured to efficiently process bone, cartilage and tissue.

These and other embodiments are described in more detail below.

### BRIEF DESCRIPTION OF THE FIGURES

FIG. 1 is a perspective view of the handheld rotary medical device.

FIG. 2 is a perspective view of the outer shaft with inner drive shaft positioned within the outer shaft, whereby the outer and inner shafts are detached from the handle.

FIG. 3 is an exploded, perspective view of the outer shaft and inner drive shaft of FIG. 2.

FIG. 4 is an exploded, perspective view of the outer shaft and inner drive shaft of FIG. 2 with a longitudinal axis of the inner drive shaft aligned with a longitudinal axis of the outer shaft.

FIG. 5 is a cross-sectional, exploded, perspective view of the outer shaft and inner drive shaft taken along Section Line 5-5 in FIG. 4.

FIG. 6 is a cross-sectional, perspective view of the outer shaft and inner drive shaft taken along Section Line 6-6 in FIG. 2.

FIG. 7 is a perspective view of the handheld rotary medical device including a control console, foot controls and handle.

FIG. 8 is a perspective view of an autologous tissue collector configure to be attached downstream of the inner and outer shafts to capture harvested material.

### DETAILED DESCRIPTION OF THE FIGURES

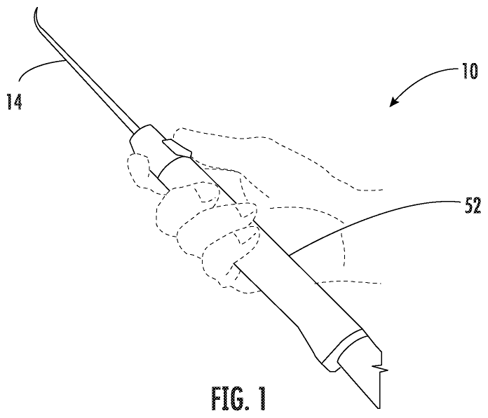

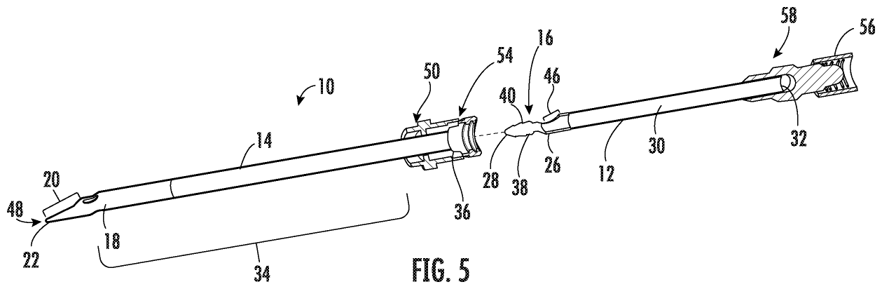

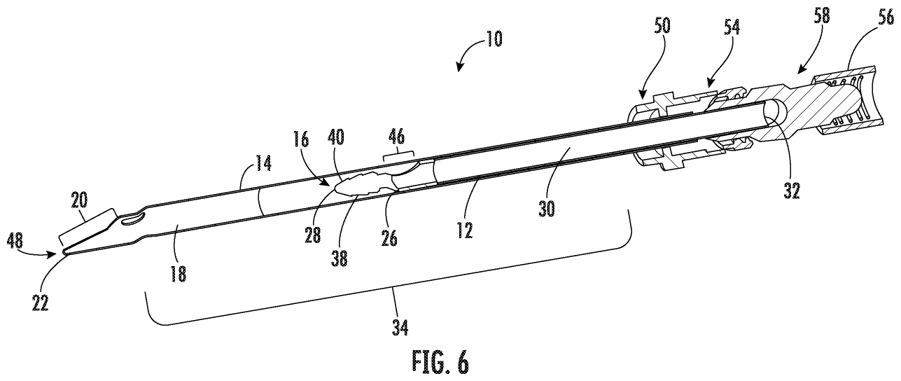

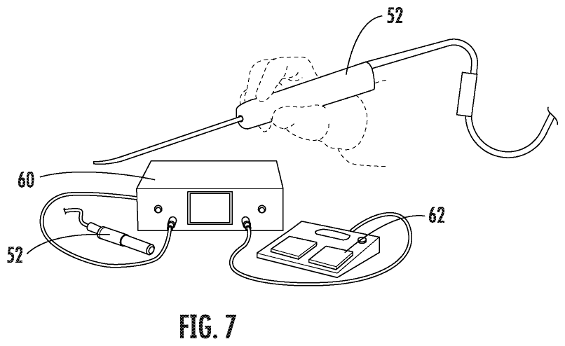

As shown in FIGS. 1-8, a handheld rotary medical device 10 configured to harvest material from a patient and create particulates of the material within an inner drive shaft 12 is disclosed. In at least one embodiment, the device 10 may include a rotatable inner drive shaft 12 positioned within an elongated, tubular, outer shaft 14, whereby the inner drive shaft 12 includes an inner drive shaft working member 16 configured to reduce excised material within a channel 18 created by the outer shaft 14 from material, such as tissue, drawn through a distal opening 20 in the outer shaft 14 before the reduce excised material is evacuated proximally therefrom. As such, the material collected at a distal end 22 of an outer shaft 14 is processed via an inner drive shaft working member 15 before the material is evacuated out of the outer shaft 14 and into a material collection system 24.

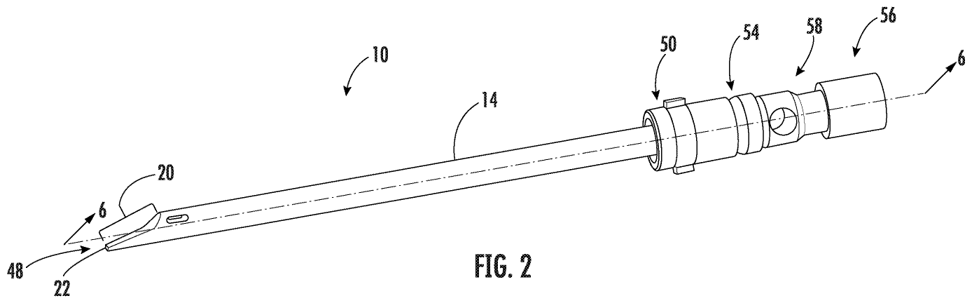

In at least one embodiment, as shown in FIGS. 2-6, the handheld rotary medical device 10 may be formed from an inner drive shaft 12 configured to be positioned within an elongated, tubular, outer shaft 14. The outer shaft 14 may encapsulate the inner drive shaft 12 such that the inner drive shaft 12 is positioned within the outer shaft 14. The inner drive shaft 12 may be formed with a generally elongated configuration. In at least one embodiment, the inner drive shaft 12 may be a generally elongated, tubular shaft with an inner evacuation channel 30 extending through at least a portion of the inner drive shaft 12, and in at least one embodiment, from a distal end 26 of the inner drive shaft 12 to a proximal end 32 of the inner drive shaft 12. The inner drive shaft 12 may be formed from a resilient material, such as, but not limited to, stainless steel. The outer shaft 14 may be a generally elongated, tubular shaft with an inner evacuation channel 18 extending through at least a portion of the outer shaft 14, and in at least one embodiment, from a distal end 34 of the outer shaft 14 to a proximal end 36 of the outer shaft 12. The inner drive shaft 12 may be formed from a resilient material, such as, but not limited to, stainless steel. The inner drive shaft 12 may be sized to fit within the channel 18 created by the outer shaft 14, whereby an outer diameter of the inner drive shaft 12 is less than an inner diameter of the outer shaft 14.

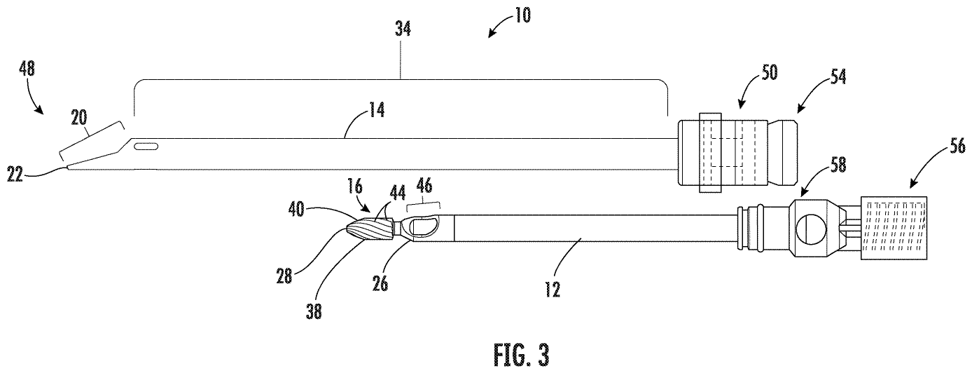

The device 10 may include an inner drive shaft working member 16 at a distal end 26 of the inner drive shaft 12, as shown in FIGS. 3-6. The inner drive shaft working member 16 may be positioned such that a distalmost point 28 of the inner drive shaft working member 16 resides within a channel 18 created by the outer shaft 14 and is offset proximally from a distal end 22 of the outer shaft 22. The inner drive shaft working member 16 may be configured to reduce excised material within the channel 18 created by the outer shaft 14 from tissue drawn through a distal opening 20 in the outer shaft 14 before the reduce excised material is evacuated proximally therefrom. The inner drive shaft working member 16 may be formed from any shape and configuration desired to yield a desired end product of harvested material. As such, the device 10 may include one or multiple inner drive shaft working members 16 having different configurations, such as different, sizes or shapes or both. The inner drive shaft working member 16 may be releasably or permanently attached to the inner drive shaft 12.

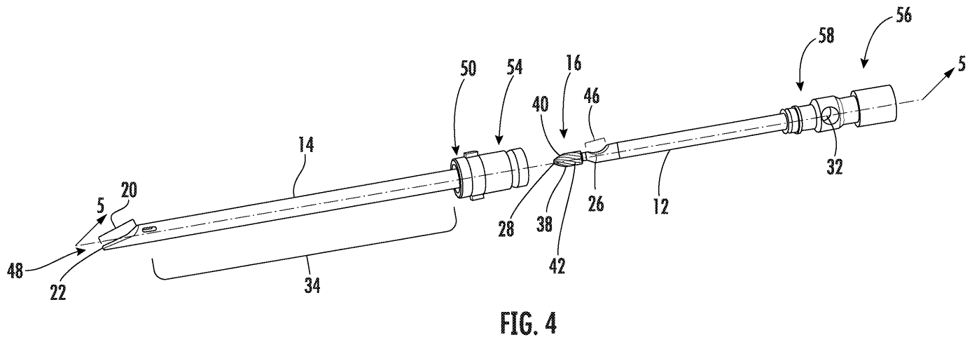

In at least one embodiment, as shown in FIGS. 3-6, the inner drive shaft working member 16 may be formed in the shape of a bulbous cone with one or more material engaging members 38 on an outer surface 40 thereof for engaging material, such as, but not limited to, tissue, cartilage and bone. In at least one embodiment, as shown in FIG. 4, the material engaging member 38 may include a plurality of burrs 42 configured for engaging material. In another embodiment, as shown in FIG. 3, the material engaging member 38 may include a plurality of teeth 44 extending radially outward from the outer surface 40 of the inner drive shaft working member 16. The plurality of teeth 44 may be aligned into a plurality of rows. The rows of teeth 44 may extend circumferentially about the inner drive shaft working member 16, which may be orthogonal to a longitudinal axis of the inner drive shaft 12, aligned with the longitudinal axis of the inner drive shaft 12 or nonparallel and nonorthogonal to the longitudinal axis of the inner drive shaft 12, or any combination thereof.

The device 10 may include one or more openings 46 in the inner drive shaft 12, as shown in FIGS. 3-6, for capturing reduced excised material after passing the inner drive shaft working member 16 in the channel 18 created by the outer shaft 14. The opening 46 in the inner drive shaft 12 may be positioned near the distal end 26 of the inner drive shaft 12 and proximal of the inner drive shaft working member 16.

The device 10 may include one or more outer shaft working members 48 positioned at a distal end 34 of the outer shaft 14 and configured to capture material so that the material can be evacuated proximally therefrom into the channel 18 in the outer shaft 14. In at least one embodiment, the outer shaft working member 48 may be a sharp freer configured to excise tissue. In another embodiment, the outer shaft working member 48 may be a curette configured to excise tissue. In another embodiment, the outer shaft working member 48 may be a shaver.

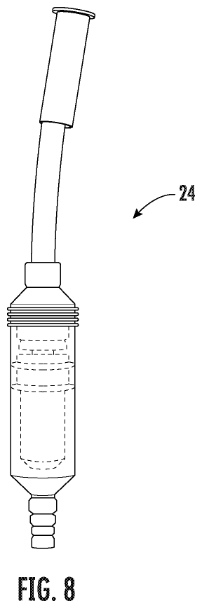

The device 10 may include one or more material collection systems 24, as shown in FIG. 8, in fluid communication with the inner drive shaft 12 to evacuate material from the outer shaft 14. In at least one embodiment, the device 10 may include one or more material collection systems 24 in fluid communication with the inner evacuation channel 30 of the inner drive shaft 12 to evacuate material through the inner drive shaft 12. The material collection system 24 may include one or more containers configured to capture and retain material removed from a patient. In at least one embodiment, the material collection system 24 may include one or more autologous tissue collectors, such as, but not limited to, a GRAFTNET device, available from Arthrex, Inc., Naples, Florida.

The device 10, as shown in FIGS. 2-6, may include one or more outer shaft bases 50 configured to be removably coupled directly or indirectly to a handle 52. The handle 52 may be formed have any appropriate configuration enabling a user, such as, but not limited to, a healthcare professional, to singlehandedly grasp the handle 52 to control operation of the device 10. The handle 52 may include one or more switches to control operation of the device 10. The device 10 may include one or more releasable connection systems 54 configured to enable the outer shaft 14 to be releasably attachable to the handle 52 of the medical device 10. The device 10 may include one or more inner shaft bases 58 which in turn may include one or more releasable connections 56 configured to be releasably attachable to a handle 52 of the device 10 to enable the inner drive shaft 12 to be releasably attachable to the handle 52 of the medical device 10. The releasable connection system 54 of the outer shaft 14 and the releasable connection system 56 of the inner drive shaft 12 may be any appropriate system enabling the outer shaft 14 and inner drive shaft 12 to be releasably attachable to the handle 52. The outer shaft 14 or inner shaft 12, or both, may be attachable to the material collection system 24.

During use, if not already assembled, the inner drive shaft 12 may be inserted into the channel 18 formed by the outer shaft 14 such that the releasable connector 56 of the inner drive shaft 12 contact the outer shaft base 50. In such position, the inner drive shaft working member 16 may be positioned within the channel 18 of the outer shaft 14. The inner drive shaft working member 16 may be positioned such that the distalmost point 28 of the inner drive shaft working member 16 resides within the channel 18 created by the outer shaft 14 generally in a midsection 34 of the outer shaft 14 between distal and proximal ends 22, 36, respectively, of the outer shaft 14. The outer shaft working member 48 may be advanced to contact material, such as, but not limited to, tissue, bone and cartilage within a patient. The working member 48 may excise material from the patient, whereby at least a portion of the excised material, if not all of the excised material, may be drawn into the distal opening 20 and into the channel 18 inside the outer shaft 14. The excised material may continue to be drawn proximally via suction forces and drawn into contact with the inner drive shaft working member 16. The inner driver shaft 12 rotates during use to process the excised material. A user may control the control parameters of the inner drive shaft 12 via a control console 60 and other devices, such as foot pedals 62. In at least one embodiment, the handheld rotary medical device 10 may be configured to enable a user to control the rotational speed of the inner driver shaft 12. The operational settings of the handheld rotary medical device 10 may be adjustable to control the size and shape of particulate matter created, harvested and collected by the handheld rotary medical device 10. The operational settings of the inner drive shaft 12 may include, but are not limited to, rotational speed, single rotational direction operation, multiple rotational direction operation, oscillation speed, torque control, momentum control and the like. The operational settings of the inner drive shaft 12 may be controlled by a user to control the size and shape of the particulate matter created with the rotational action of the inner drive shaft 12.

The size and shape of the particulate matter may also be controlled by changing the shape of the inner drive shaft working member 16. In particular, the inner drive shaft working member 16 may have different shapes and configurations, such as, but not limited to, a bulbous cone, as shown in FIGS. 3-6, and different sizes depending on the desired size and shape of the output particulate matter of the handheld rotary medical device 10. The handheld rotary medical device 10 may include one or a plurality of inner drive shafts 12 with differently configured inner drive shaft working members 16. In at least one embodiment, the inner drive shafts 12 with differently configured inner drive shaft working members 16 may be interchangeable. Thus, there may be numerous different handheld rotary medical devices 10, each having different inner drive shaft working members 16 or numerous different inner drive shafts 12 with different inner drive shaft working members 16 configured to be removably positioned within the outer shaft 14. In another embodiment, the inner drive shaft working member 16 may be removably attached to the inner drive shaft 12 such that a user can change the inner drive shaft working member 16 to yield a desired size and shape of the particulate matter created with the rotational action of the inner drive shaft 12.

When the excised material contacts the inner drive shaft working member 16, the material engaging members 38 of the inner drive shaft working member 16 reduce the excised material 38 as the excised material passes the inner drive shaft working member 16 in the channel 18. The excised material then flows through the opening 46 and into the inner evacuation channel 30 in the inner drive shaft 12. The excised material continues through the inner evacuation channel 30 in the inner drive shaft 12 and is passed onto a material collection system 24, which may be positioned in the handle 52 or elsewhere. The reduced material may then be processed as desired.

The foregoing is provided for purposes of illustrating, explaining, and describing embodiments. Modifications and adaptations to these embodiments will be apparent to those skilled in the art.

We claim:

1. A handheld rotary medical device, comprising:
an inner drive shaft;
an elongated, tubular, outer shaft encapsulating the inner drive shaft such that the inner drive shaft is positioned within the outer shaft;
an inner drive shaft working member at a distal end of the inner drive shaft, wherein a distalmost point of the inner drive shaft working member resides within a channel created by the outer shaft and is offset proximally from a distal end of the outer shaft; and
wherein the inner drive shaft working member is configured to reduce excised material within the channel created by the outer shaft from tissue drawn through a distal opening in the outer shaft before the reduced excised material is evacuated proximally therefrom.

2. The medical device of claim 1, wherein the inner drive shaft working member is a bulbous cone with at least one material engaging member on an outer surface thereof for engaging material.

3. The medical device of claim 1, wherein the at least one material engaging member on an outer surface of the inner drive shaft working member comprises a plurality of burrs configured for engaging material.

4. The medical device of claim 1, wherein the at least one material engaging member on an outer surface of the inner drive shaft working member comprises a plurality of teeth extending radially outward from the outer surface of the inner drive shaft working member, wherein the plurality of teeth are aligned into rows.

5. The medical device of claim 1, further comprising at least one opening in the inner drive shaft for capturing reduced excised material after passing the inner drive shaft working member in the channel created by the outer shaft.

6. The medical device of claim 5, wherein the at least one opening in the inner drive shaft is near the distal end of the inner drive shaft and proximal of the inner drive shaft working member.

7. The medical device of claim 1, further comprising an outer shaft working member positioned at a distal end of the outer shaft and configured to capture material so that the material can be evacuated proximally therefrom.

8. The medical device of claim 7, wherein the outer shaft working member comprises a sharp freer configured to excise tissue.

9. The medical device of claim 7, wherein the outer shaft working member comprises a curette configured to excise tissue.

10. The medical device of claim 7, wherein the outer shaft working member comprises a shaver.

11. The medical device of claim 1, further comprising at least one material collection system in fluid communication with the inner drive shaft to evacuate material through the inner drive shaft.

12. The medical device of claim 1, further comprising at least one outer shaft base comprising at least one releasable connection configured to be releasably attachable to a handle of the medical device.

13. The medical device of claim 1, further comprising at least one inner housing base comprising at least one releasable connection configured to be releasably attachable to a handle of the medical device.

14. A handheld rotary medical device, comprising:
an inner drive shaft;
an elongated, tubular, outer shaft encapsulating the inner drive shaft such that the inner drive shaft is positioned within the outer shaft;
an inner drive shaft working member at a distal end of the inner drive shaft, wherein a distalmost point of the inner drive shaft working member resides within a channel created by the outer shaft and is offset proximally from a distal end of the outer shaft;
at least one opening in the inner drive shaft for capturing reduced excised material after passing the inner drive shaft working member in the channel created by the outer shaft; and
an outer shaft working member positioned at a distal end of the outer shaft and configured to capture material so that the material can be evacuated proximally therefrom; and
wherein the inner drive shaft working member is configured to reduce excised material within the channel created by the outer shaft from tissue drawn through a distal opening in the outer shaft before the reduced excised material is evacuated proximally therefrom.

15. The medical device of claim 14, wherein the inner drive shaft working member is a bulbous cone with at least one material engaging member on an outer surface thereof for engaging material.

16. The medical device of claim 14, wherein the at least one material engaging member on an outer surface of the inner drive shaft working member comprises a plurality of burrs configured for engaging material.

17. The medical device of claim 14, wherein the at least one material engaging member on an outer surface of the inner drive shaft working member comprises a plurality of teeth extending radially outward from the outer surface of the inner drive shaft working member, wherein the plurality of teeth are aligned into rows.

18. The medical device of claim 14, wherein the at least one opening in the inner drive shaft is near the distal end of the inner drive shaft and proximal of the inner drive shaft working member.

19. The medical device of claim 14, wherein the outer shaft working member comprises a sharp freer configured to excise tissue.

20. The medical device of claim 14, wherein the outer shaft working member comprises a curette configured to excise tissue.

21. The medical device of claim 14, wherein the outer shaft working member comprises a shaver.

22. The medical device of claim 14, further comprising at least one material collection system in fluid communication with the inner drive shaft to evacuate material through the inner drive shaft.

23. The medical device of claim 14, further comprising at least one outer shaft base comprising at least one releasable connection configured to be releasably attachable to a handle of the medical device.

24. The medical device of claim 14, further comprising at least one inner housing base comprising at least one releasable connection configured to be releasably attachable to a handle of the medical device.

25. A handheld rotary medical device, comprising:
an inner drive shaft;
an elongated, tubular, outer shaft encapsulating the inner drive shaft such that the inner drive shaft is positioned within the outer shaft;
an inner drive shaft working member at a distal end of the inner drive shaft, wherein a distalmost point of the inner drive shaft working member resides within a channel created by the outer shaft and is offset proximally from a distal end of the outer shaft;
at least one opening in the inner drive shaft for capturing reduced excised material after passing the inner drive shaft working member in the channel created by the outer shaft;
at least one material collection system in fluid communication with the inner drive shaft to evacuate material through the inner drive shaft; and
wherein the inner drive shaft working member is configured to reduce excised material within the channel created by the outer shaft from tissue drawn through a distal opening in the outer shaft before the reduced excised material is evacuated proximally therefrom.

26. The medical device of claim 25, wherein the inner drive shaft working member is a bulbous cone with at least one material engaging member on an outer surface thereof for engaging material.

27. The medical device of claim 25, wherein the at least one material engaging member on an outer surface of the inner drive shaft working member comprises a plurality of burrs configured for engaging material.

28. The medical device of claim 25, wherein the at least one material engaging member on an outer surface of the inner drive shaft working member comprises a plurality of teeth extending radially outward from the outer surface of the inner drive shaft working member, wherein the plurality of teeth are aligned into rows.

29. The medical device of claim 25, wherein the at least one opening in the inner drive shaft is near the distal end of the inner drive shaft and proximal of the inner drive shaft working member.

30. The medical device of claim 25, further comprising an outer shaft working member positioned at a distal end of the outer shaft and configured to capture material so that the material can be evacuated proximally therefrom.

31. The medical device of claim 25, further comprising at least one outer shaft base comprising at least one releasable connection configured to be releasably attachable to a handle of the medical device.

32. The medical device of claim 25, further comprising at least one inner housing base comprising at least one releasable connection configured to be releasably attachable to a handle of the medical device.
